(19) United States
(12) Patent Application Publication
(10) Pub. No.: US 2018/0153976 A1
(43) Pub. Date: Jun. 7, 2018

(54) NOVEL CMV PP65 TARGETING DNA VACCINE FOR CANCER IMMUNOTHERAPY

(71) Applicant: VAXIMM AG, Basel (CH)

(72) Inventor: Heinz Lubenau, Neustadt an der Weinstrasse (DE)

(21) Appl. No.: 15/737,679

(22) PCT Filed: Jun. 6, 2016
(86) PCT No.: PCT/EP2016/001003

§ 371 Date: Dec. 18, 2017

(30) Foreign Application Priority Data
Jun. 18, 2015 (EP) 15001802.6

(51) Int. Cl. A61K 39/00; C12N 1/36; C07K 14/005; A61K 39/245; A61P 31/22; A61P 35/00; A61K 45/06

(52) U.S. Cl. CPC A61K 39/0011; C12N 1/36; C07K 14/005; A61K 39/245; A61P 31/22; A61P 35/00; A61K 2039/542; C12N 2710/16122; C12N 2710/16134; A61K 2039/522; A61K 2039/523; A61K 2039/585; A61K 45/06

(57) ABSTRACT

The present invention relates to an attenuated strain of Salmonella comprising a DNA molecule encoding CMV pp65. In particular, the present invention relates to the use of said attenuated strain of Salmonella in cancer immunotherapy.

### FIELD OF THE INVENTION

The present invention relates to an attenuated strain of Salmonella comprising a DNA molecule encoding CMV pp65. In particular, the present invention relates to the use of said attenuated strain of Salmonella in cancer immunotherapy.

### BACKGROUND OF THE INVENTION

Human Cytomegalovirus (HCMV) proteins and oligonucleotides are expressed in a high percentage of gliomas. One study detected the HCMV immediate early 1 (IE1) protein in 100% of glioblastomas and 82% of low-grade gliomas using immunohistochemistry. Further studies have identified the presence of the HCMV proteins IE1, US28, pp65, gB, HCMV IL-10, and pp28 and the HCMV genes IE1 and gB. HCMV sequences and viral gene expression exist in most, if not all, malignant gliomas. In contrast, no HCMV proteins or nucleotides were detected in normal brain controls or areas of normal brain adjacent to tumor.

At present, HCMV is not considered to be an oncogenic virus as it does not seem to be directly involved in transformation. However, increasing evidence suggests that HCMV can modulate the malignant phenotype in glioblastomas. There is a significant overlap of HCMV biology with the essential alterations of cell physiology that are hallmarks of cancer supporting HCMV's role as oncomodulator, such as sustaining proliferative signaling, evading growth suppressors, activating invasion and metastasis, enabling replicative immortality, inducing angiogenesis, resisting cell death, deregulating cellular energetics, avoiding immune destruction, genome instability and mutation and tumor promoting inflammation.

A major immunodominant protein of human cytomegalovirus (CMV) is the tegument protein CMV pp65. The biologic function of CMV pp65 is unclear, but it is believed to be involved in cell cycle regulation. CMV pp65 is a nucleotropic protein which is able to bind polo-like kinase 1 (PLK-1) and has been shown to have protein kinase activity.

Recent evidence supports the development of therapeutic HCMV pp65 vaccines to reduce glioblastoma's malignancy. Upon vaccination with autologous dendritic cells pulsed with autologous tumor lysate in a phase I clinical trial, one patient developed a robust HCMV-specific CD8+ T-cell response to the pp65 HCMV immunodominant epitope that began immediately after one injection of autologous tumor lysate-pulsed dendritic cells.

Thus HCMV pp65 has emerged as promising candidate for cancer immunotherapy, based on its immunogenic potential and its favorable expression pattern in cancer patients.

Attenuated derivatives of Salmonella enterica are attractive vehicles for the delivery of heterologous antigens to the mammalian immune system, since S. enterica strains can potentially be delivered via mucosal routes of immunization, i.e. orally or nasally, which offers advantages of simplicity and safety compared to parenteral administration. Furthermore, Salmonella strains elicit strong humoral and cellular immune responses at the level of both systemic and mucosal compartments. Batch preparation costs are relatively low and formulations of live bacterial vaccines are highly stable. Attenuation can be accomplished by deletion of various genes, including virulence, regulatory, and metabolic genes.

Several Salmonella typhimurium strains attenuated by aro mutations have been shown to be safe and effective delivery vehicles for heterologous antigens in animal models.

Approaches of delivering DNA constructs encoding antigens, in particular the tumor stroma antigen VEGFR, via live attenuated Salmonella typhimurium strains into mouse target cells are described in WO 03/073995. Niethammer et al., (Nature Medicine 2002, 8(12), 1369) demonstrated that the attenuated S. typhimurium aroA strain SL7207 harboring an expression vector encoding the murine vascular endothelial growth factor receptor 2 (VEGFR-2 or FLK-1), which is essential for tumor angiogenesis, is functional as a cancer vaccine.

There is however only one attenuated Salmonella enterica serovar strain, namely Salmonella enterica serovar typhi Ty21a (short: S. typhi Ty21a), which has been accepted for use in humans and is distributed under the trade name of Vivotif® (Berna Biotech Ltd., a Crucell Company, Switzerland; marketing authorization number PL 15747/0001 dated 16 Dec. 1996).

This well-tolerated, live oral vaccine against typhoid fever was derived by chemical mutagenesis of the wild-type virulent bacterial isolate S. typhi Ty2 and harbors a loss-of-function mutation in the galE gene, as well as other less defined mutations. It has been licensed as typhoid vaccine in many countries after it was shown to be efficacious and safe in field trials.

WO 2014/005683 discloses an attenuated strain of Salmonella comprising a recombinant DNA molecule encoding a VEGF receptor protein for use in cancer immunotherapy, particularly for use in the treatment of pancreatic cancer.

WO 2014/173542 discloses an attenuated strain of Salmonella comprising a recombinant DNA molecule encoding Wilms' Tumor Protein (WT1) for use in cancer immunotherapy.

WO 2013/091898 discloses a method for growing attenuated mutant Salmonella typhi strains lacking galactose epimerase activity and harboring a recombinant DNA molecule.

CMV pp65 is a promising tumor-specific viral antigen for the development of cancer vaccines. The great need for improved cancer therapy approaches based on targeting CMV pp65 has not been met so far.

### OBJECTS OF THE INVENTION

In view of the prior art, it is an object of the present invention to provide a novel oral CMV pp65 targeting cancer vaccine. Such a CMV pp65 targeting cancer vaccine would offer major advantages for improving the treatment options for cancer patients.

### SUMMARY OF THE INVENTION

In one aspect, the present invention relates to an attenuated strain of Salmonella comprising at least one copy of a DNA molecule comprising an expression cassette encoding CMV pp65.

The attenuated Salmonella strain of the present invention may elicit strong immune responses. To the inventor's knowledge, this novel attenuated Salmonella strain is the first oral cancer vaccine targeting CMV pp65. Since CMV pp65 is expressed in more than 90% of glioblastoma specimens but not in the surrounding normal brain tissue, the attenuated Salmonella strain of the present invention has great potential as cancer vaccine for the treatment of glioblastomas.

The vaccine according to the present invention (VXM65) may elicit strong CMV pp65-specific immune responses. Vaccination with VXM65 may lead to an immune response and the development of an immune memory against tumor cells harbouring CMV pp65 protein. It is remarkable and surprising that the novel vaccine VXM65 is effective at relatively low doses. The attenuated Salmonella strain of the present invention may be applied in monotherapy or in combination with a second attenuated strain of Salmonella comprising a DNA molecule encoding a second tumor antigen. Furthermore, the attenuated strain of the present invention may be administered in combination with chemotherapy, radiotherapy or biological cancer therapy. Treatment with VXM65 may also be effective, if the patient has developed a resistance to chemotherapy (chemo-refractory patients). The novel attenuated Salmonella strain of the present invention might therefore be useful in novel, greatly improved cancer therapy approaches.

In particular embodiments, the attenuated strain of Salmonella is of the species Salmonella enterica. In particular embodiments, the attenuated strain of Salmonella is Salmonella typhi Ty21a.

In particular embodiments, the expression cassette is a eukaryotic expression cassette.

In particular embodiments, CMV pp65 is selected from the group consisting of human CMV pp65 having the amino acid sequence as found in SEQ ID NO 1 and a protein that shares at least 80% sequence identity therewith.

In particular other embodiments, CMV pp65 is selected from the group consisting of human CMV pp65 having the amino acid sequence as found in SEQ ID NO 2 and a protein that shares at least 80% sequence identity therewith.

In particular other embodiments, CMV pp65 is selected from the group consisting of human CMV pp65 having the amino acid sequence as found in SEQ ID NO 3 and a protein that shares at least 80% sequence identity therewith.

In particular embodiments, the CMV pp65 has the amino acid sequence as found in SEQ ID NO 1.

In particular other embodiments, the CMV pp65 has the amino acid sequence as found in SEQ ID NO 2.

In particular other embodiments, the CMV pp65 has the amino acid sequence as found in SEQ ID NO 3.

In particular embodiments, the DNA molecule comprises the kanamycin antibiotic resistance gene, the pMB1 ori, and a eukaryotic expression cassette encoding CMV pp65 having the amino acid sequence as found in SEQ ID NO 1 or a protein that shares at least 80% sequence identity therewith, under the control of a CMV promoter. In particular embodiments, CMV pp65 has the nucleic acid sequence as found in SEQ ID NO 4.

In particular embodiments, the DNA molecule comprises the kanamycin antibiotic resistance gene, the pMB1 ori, and a eukaryotic expression cassette encoding CMV pp65 having the amino acid sequence as found in SEQ ID NO 2 or a protein that shares at least 80% sequence identity therewith, under the control of a CMV promoter. In particular embodiments, CMV pp65 has the nucleic acid sequence as found in SEQ ID NO 5.

In particular embodiments, the DNA molecule comprises the kanamycin antibiotic resistance gene, the pMB1 ori, and a eukaryotic expression cassette encoding CMV pp65 having the amino acid sequence as found in SEQ ID NO 3 or a protein that shares at least 80% sequence identity therewith, under the control of a CMV promoter. In particular embodiments, CMV pp65 has the nucleic acid sequence as found in SEQ ID NO 6.

In particular embodiments, the attenuated strain of Salmonella is for use as a medicament.

In particular embodiments, the attenuated strain of Salmonella is for use as a vaccine.

In particular embodiments, the attenuated strain of Salmonella is for use in cancer immunotherapy.

In particular embodiments, cancer immunotherapy further comprises administration of one or more further attenuated strain(s) of Salmonella comprising at least one copy of a DNA molecule comprising an expression cassette encoding a tumor antigen and/or a tumor stroma antigen. In particular embodiments, said one or more further attenuated strain(s) of Salmonella is/are Salmonella typhi Ty21a comprising a eukaryotic expression cassette. In particular embodiments, said one or more further strain(s) of Salmonella comprise(s) an attenuated strain(s) of Salmonella encoding the tumor stroma antigen human VEGFR-2 and/or the tumor antigen human Wilms' Tumor Protein (WT1) and/or the tumor antigen human Mesothelin (MSLN) and/or the tumor antigen human CEA.

In particular embodiments, cancer immunotherapy further comprises administration of one further attenuated strain of Salmonella, in particular Salmonella typhi Ty21a, comprising at least one copy of a DNA molecule comprising an expression cassette, in particular a eukaryotic expression cassette, encoding a tumor antigen or a tumor stroma antigen. In particular embodiments said tumor antigen or tumor stroma antigen encoded by said further attenuated strain of Salmonella is selected from human VEGFR-2, human Wilms' Tumor Protein (WT1), human Mesothelin (MSLN) and human CEA.

In particular embodiments, the attenuated strain of Salmonella is co-administered with said one or more further attenuated strain(s) of Salmonella.

In particular embodiments, cancer immunotherapy is accompanied by chemotherapy, radiotherapy or biological cancer therapy.

In particular embodiments, the attenuated strain of Salmonella is administered during the chemotherapy or the radiotherapy treatment cycle or during biological cancer therapy.

In particular embodiments, the attenuated strain of Salmonella is administered before the chemotherapy or the radiotherapy treatment cycle or before biological cancer therapy.

In particular embodiments, the attenuated strain of Salmonella is administered after the chemotherapy or the radiotherapy treatment cycle or after biological cancer therapy.

In further embodiments the attenuated strain of Salmonella is administered before and during at least one of the chemotherapy, the radiotherapy treatment cycle and the biological cancer therapy. In cases where more than one of the chemotherapy, the radiotherapy and the biological cancer therapy are carried out the attenuated strain of Salmonella may be administered before or during or before and during at least one of these therapies, particularly during at least two of these therapies.

In particular embodiments, the attenuated strain of Salmonella is administered orally.

In particular embodiments, the cancer is selected from gliomas, in particular from glioblastomas.

In particular embodiments, the single dose is from about 10⁵ to about 10¹¹, particularly from about 10⁶ to about 10¹⁰, more particularly from about 10⁶ to about 10⁹, more particularly from about 10⁶ to about 10⁸, most particularly from about 10⁶ to about 10⁷ colony forming units (CFU).

In particular embodiments, the attenuated strain of Salmonella is for use in personalized cancer immunotherapy comprising the step of assessing the CMV pp65 expression pattern and/or the pre-immune response against CMV pp65 of a patient.

### DETAILED DESCRIPTION OF THE INVENTION

The present invention may be understood more readily by reference to the following detailed description of the invention and the examples included therein.

In one aspect, the present invention relates to an attenuated strain of Salmonella comprising at least one copy of a DNA molecule comprising an expression cassette encoding CMV pp65, in particular pp65 of human CMV.

According to the invention, the attenuated Salmonella strain functions as the bacterial carrier of the DNA molecule comprising an expression cassette encoding CMV pp65 for the delivery of said DNA molecule into a target cell.

In the context of the present invention, the term “attenuated” refers to a bacterial strain of reduced virulence compared to the parental bacterial strain, not harboring the attenuating mutation. Attenuated bacterial strains have preferably lost their virulence but retained their ability to induce protective immunity. Attenuation can be accomplished by deletion of various genes, including virulence, regulatory, and metabolic genes. Attenuated bacteria may be found naturally or they may be produced artificially in the laboratory, for example by adaptation to a new medium or cell culture or they may be produced by recombinant DNA technology. Administration of about 10¹¹ CFU of the attenuated strain of Salmonella according to the present invention preferably causes Salmonellosis in less than 5%, more preferably less than 1%, most preferably less than 1% of subjects.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment the term “comprising” as used throughout the application and in particular within the claims may be replaced by the term “consisting of”.

The DNA molecule comprising an expression cassette encoding CMV pp65 is suitably a recombinant DNA molecule, i.e. an engineered DNA construct, preferably composed of DNA pieces of different origin. The DNA molecule can be a linear nucleic acid, or preferably, a circular DNA plasmid generated by introducing an open reading frame encoding CMV pp65 into an expression vector plasmid.

In the context of the present invention, the term “expression cassette” refers to a nucleic acid unit comprising at least the CMV pp65 gene under the control of regulatory sequences controlling its expression. The expression cassette comprised in the attenuated strain of Salmonella can preferably mediate transcription of the included open reading frame encoding CMV pp65 in a target cell. Expression cassettes typically comprise a promoter, at least one open reading frame and a transcription termination signal.

The tegument protein CMV pp65 is a major immunodominant protein of human cytomegalovirus (CMV). The biologic function of CMV pp65 is unclear, but it is believed to be involved in cell cycle regulation. CMV pp65 is a nucleotropic protein exhibiting protein kinase activity, which is able to bind polo-like kinase 1 (PLK-1).

HCMV pp65 is expressed in more than 90% of glioblastoma specimens but not in surrounding normal brain. This viral protein is thus a promising candidate as tumor-specific target for the development novel of cancer immunotherapies.

The CMV pp65 protein contains two bipartite nuclear localization signals (NLSs) at amino acids 415 to 438 and amino acids 537 to 561 near the carboxy terminus and a phosphate binding site related to its kinase activity at lysine-436. Mutating the lysine at position 436 to asparagine and deletion of amino acids 537 to 561 results in a protein without kinase activity and markedly reduced nuclear localization. This mutant protein exhibits unaltered immunogenicity.

In particular embodiments, the attenuated strain of Salmonella is of the species Salmonella enterica. In particular embodiments, the attenuated strain of Salmonella is Salmonella typhi Ty21a. The attenuated S. typhi Ty21a strain is the active component of Typhoral L®, also known as Vivotif® (manufactured by Berna Biotech Ltd., a Crucell Company, Switzerland). It is currently the only licensed live oral vaccine against typhoid fever. This vaccine has been extensively tested and has proved to be safe regarding patient toxicity as well as transmission to third parties (Wandan et al., J. Infectious Diseases 1982, 145:292-295). The vaccine is licensed in more than 40 countries. The Marketing Authorization number of Typhoral L® is PL 15747/0001 dated 16 Dec. 1996. One dose of vaccine contains at least 2×10⁹ viable S. typhi Ty21a colony forming units and at least 5×10⁹ non-viable S. typhi Ty21a cells.

One of the biochemical properties of the Salmonella typhi Ty21a bacterial strain is its inability to metabolize galactose. The attenuated bacterial strain is also not able to reduce sulfate to sulfide which differentiates it from the wild-type Salmonella typhi Ty2 strain. With regard to its serological characteristics, the Salmonella typhi Ty21a strain contains the 09-antigen which is a polysaccharide of the outer membrane of the bacteria and lacks the 05-antigen which is in turn a characteristic component of Salmonella typhimurium. This serological characteristic supports the rationale for including the respective test in a panel of identity tests for batch release.

In particular embodiments, the expression cassette is a eukaryotic expression cassette. In the context of the present invention, the term “eukaryotic expression cassette” refers to an expression cassette which allows for expression of the open reading frame in a eukaryotic cell. It has been shown that the amount of heterologous antigen required to induce an adequate immune response may be toxic for the bacterium and result in cell death, over-attenuation or loss of expression of the heterologous antigen. Using a eukaryotic expression cassette that is not expressed in the bacterial vector but only in the target cell may overcome this toxicity problem and the protein expressed may exhibit a eukaryotic glycosylation pattern.

A eukaryotic expression cassette comprises regulatory sequences that are able to control the expression of an open reading frame in a eukaryotic cell, preferably a promoter and a polyadenylation signal. Promoters and polyadenylation signals included in the recombinant DNA molecules comprised by the attenuated strain of Salmonella of the present invention are preferably selected to be functional within the cells of the subject to be immunized. Examples of suitable promoters, especially for the production of a DNA vaccine for humans, include but are not limited to promoters from Cytomegalovirus (CMV), such as the strong CMV immediate early promoter, Simian Virus 40 (SV40), Mouse Mammary Tumor Virus (MMTV), Human Immunodeficiency Virus (HIV), such as the HIV Long Terminal Repeat (LTR) promoter, Moloney virus, Epstein Barr Virus (EBV), and from Rous Sarcoma Virus (RSV) as well as promoters from human genes such as human actin, human myosin, human hemoglobin, human muscle creatine, and human metallothionein. In a particular embodiment, the eukaryotic expression cassette contains the CMV promoter. In the context of the present invention, the term “CMV promoter” refers to the strong immediate-early cytomegalovirus promoter.

Examples of suitable polyadenylation signals, especially for the production of a DNA vaccine for humans, include but are not limited to the bovine growth hormone (BGH) polyadenylation site, SV40 polyadenylation signals and LTR polyadenylation signals. In a particular embodiment, the eukaryotic expression cassette included in the DNA molecule comprised by the attenuated strain of Salmonella of the present invention comprises the BGH polyadenylation site.

In addition to the regulatory elements required for expression of the heterologous CMV pp65 gene, like a promoter and a polyadenylation signal, other elements can also be included in the recombinant DNA molecule. Such additional elements include enhancers. The enhancer can be, for example, the enhancer of human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the species to be immunized. The person skilled in the art can produce recombinant DNA molecules that are functional in a given subject species.

In particular embodiments, CMV pp65 is selected from the group consisting of CMV pp65 having the amino acid sequence as found in SEQ ID NO 1 and a protein that shares at least about 80% sequence identity therewith.

In particular other embodiments, CMV pp65 is selected from the group consisting of human CMV pp65 having the amino acid sequence as found in SEQ ID NO 2 and a protein that shares at least 80% sequence identity therewith.

In particular other embodiments, CMV pp65 is selected from the group consisting of human CMV pp65 having the amino acid sequence as found in SEQ ID NO 3 and a protein that shares at least 80% sequence identity therewith.

In this context, the term “about” or “approximately” means within 80% to 120%, alternatively within 90% to 110%, including within 95% to 105% of a given value or range.

In the context of the present invention, the term “protein that shares at least about 80% sequence identity with CMV pp65 having the amino acid sequence as found in a given SEQ ID” refers to a protein that differs in the amino acid sequence and/or the nucleic acid sequence encoding the amino acid sequence of CMV pp65 as found in the given SEQ ID. The protein may be of natural origin, e.g. a homolog of pp65 of a different species viral species, or an engineered protein, e.g. an engineered CMV pp65 derivative. It is known that the usage of codons is different between species. Thus, when expressing a heterologous protein in a target cell, it may be necessary, or at least helpful, to adapt the nucleic acid sequence to the codon usage of the target cell. Methods for designing and constructing derivatives of a given protein are well known to anyone of ordinary skill in the art.

The protein that shares at least about 80% sequence identity with CMV pp65 having a given amino acid sequence may contain one or more mutations comprising an addition, a deletion and/or a substitution of one or more amino acids, as compared to the given reference amino acid sequence. According to the teaching of the present invention, said deleted, added and/or substituted amino acids may be consecutive amino acids or may be interspersed over the length of the amino acid sequence of the protein that shares at least about 80% sequence identity with CMV pp65. According to the teaching of the present invention, any number of amino acids may be added, deleted, and/or substitutes, as long as the sequence identity with CMV pp65 is at least about 80% and the mutated CMV pp65 protein is immunogenic. Preferably, the immunogenicity of the CMV pp65 protein which shares at least about 80% sequence identity with CMV pp65 of a given amino acid sequence is reduced by less than 50%, less than 40%, less than 30%, less than 20%, less than 10%, less than 5% or less than 1% compared to the reference CMV pp65 protein of the given amino acid sequence, as measured by ELISA. Methods for designing and constructing protein homologues and for testing such homologues for their immunogenic potential are well known to anyone of ordinary skill in the art. In particular embodiments, the sequence identity with CMV pp65 having the amino acid sequence as found in SEQ ID NO 1 is at least about 80%, at least about 85%, at least about 90%, or most particularly at least about 95%. In particular embodiments, the sequence identity with CMV pp65 having the amino acid sequence as found in SEQ ID NO 2 is at least about 80%, at least about 85%, at least about 90%, or most particularly at least about 95%. In particular embodiments, the sequence identity with CMV pp65 having the amino acid sequence as found in SEQ ID NO 3 is at least about 80%, at least about 85%, at least about 90%, or most particularly at least about 95%. Methods and algorithms for determining sequence identity including the comparison of a parental protein and its derivative having deletions, additions and/or substitutions relative to a parental sequence, are well known to the practitioner of ordinary skill in the art. On the DNA level, the nucleic acid sequences encoding the protein that shares at least about 80% sequence identity with CMV pp65 of a given amino acid sequence may differ to a larger extent due to the degeneracy of the genetic code.

In particular embodiments, the CMV pp65 has the amino acid sequence as found in SEQ ID NO 1. SEQ ID NO 1 represents the amino acid sequence of wild type human CMV pp65.

In particular other embodiments, the CMV pp65 has the amino acid sequence as found in SEQ ID NO 2. SEQ ID NO 2 represents the amino acid sequence of human CMV pp65, which harbors the mutation K436N relative to the wild type human CMV pp65 of SEQ ID NO 1.

In particular other embodiments, the CMV pp65 has the amino acid sequence as found in SEQ ID NO 3. SEQ ID NO 3 represents the amino acid sequence of a truncated version of CMV pp65 of SEQ ID NO 2, which lacks the second, more C-terminal NLS (nuclear localization sequence) (i.e. amino acids 537 to 561 of CMV pp65 of SEQ ID NO 2).

In particular embodiments, the DNA molecule comprises the kanamycin antibiotic resistance gene, the pMB1 ori, and a eukaryotic expression cassette encoding CMV pp65 having the amino acid sequence as found in SEQ ID NO 1 or a protein that shares at least 80% sequence identity therewith, under the control of a CMV promoter. In particular embodiments, CMV pp65 has the nucleic acid sequence as found in SEQ ID NO 4.

In particular embodiments, the DNA molecule comprises the kanamycin antibiotic resistance gene, the pMB1 ori, and a eukaryotic expression cassette encoding CMV pp65 having the amino acid sequence as found in SEQ ID NO 2 or a protein that shares at least 80% sequence identity therewith, under the control of a CMV promoter. In particular embodiments, CMV pp65 has the nucleic acid sequence as found in SEQ ID NO 5.

In particular embodiments, the DNA molecule comprises the kanamycin antibiotic resistance gene, the pMB1 ori, and a eukaryotic expression cassette encoding CMV pp65 having the amino acid sequence as found in SEQ ID NO 3 or a protein that shares at least 80% sequence identity therewith, under the control of a CMV promoter. In particular embodiments, CMV pp65 has the nucleic acid sequence as found in SEQ ID NO 6.

In particular embodiments, the DNA molecule is a recombinant DNA molecule derived from commercially available pVAX1™ expression plasmid (Invitrogen, San Diego, Calif.). This expression vector was modified by replacing the high copy pUC origin of replication by the low copy pMB1 origin of replication of pBR322. The low copy modification was made in order to reduce the metabolic burden and to render the construct more stable. The generated expression vector backbone was designated pVAX10.

Inserting CMV pp65 with the nucleic acid sequence as found in SEQ ID NO 4 into this expression vector backbone via NheI/XhoI yielded the expression plasmid pVAX10.CMV65_1. The expression plasmid pVAX10.CMV65_1 is schematically depicted in FIG. 8. The DNA vaccine comprising the attenuated Salmonella strain Ty21a harboring the expression plasmid pVAX10.CMV65_1 is designated VXM65_1.

Inserting CMV pp65 with the nucleic acid sequence as found in SEQ ID NO 5 into the pVAX10 expression vector backbone via NheI/XhoI yielded the expression plasmid pVAX10.CMV65_2. The expression plasmid pVAX10.CMV65_2 is schematically depicted in FIG. 9. The DNA vaccine comprising the attenuated Salmonella strain Ty21a harboring the expression plasmid pVAX10.CMV65_2 is designated VXM65_2.

Inserting CMV pp65 with the nucleic acid sequence as found in SEQ ID NO 6 into the pVAX10 expression vector backbone via NheI/XhoI yielded the expression plasmid pVAX10.CMV65_3. The expression plasmid pVAX10.CMV65_3 is schematically depicted in FIG. 10. The DNA vaccine comprising the attenuated Salmonella strain Ty21a harboring the expression plasmid pVAX10.CMV65_3 is designated VXM65_3.

In particular embodiments, the attenuated strain of Salmonella is for use as a medicament.

In particular embodiments, the attenuated strain of Salmonella is for use as a vaccine.

In the context of the present invention, the term “vaccine” refers to an agent which is able to induce an immune response in a subject upon administration. A vaccine can preferably prevent, ameliorate or treat a disease. A vaccine in accordance with the present invention comprises an attenuated strain of Salmonella, preferably S. typhi Ty21a. The vaccine in accordance with the present invention further comprises at least one copy of a DNA molecule comprising an expression cassette, preferably a eukaryotic expression cassette, encoding CMV pp65, preferably selected from CMV pp65 having the amino acid sequence as found in SEQ ID NO 1 and a protein that shares at least about 80% sequence identity therewith, CMV pp65 having the amino acid sequence as found in SEQ ID NO 2 and a protein that shares at least about 80% sequence identity therewith, and CMV pp65 having the amino acid sequence as found in SEQ ID NO 3 and a protein that shares at least about 80% sequence identity therewith.

The live attenuated Salmonella mutant strain according to the present invention comprising a DNA molecule encoding CMV pp65 can be used as a vehicle for the oral delivery of this recombinant DNA molecule. Such a delivery vector comprising a DNA molecule encoding a heterologous antigen, such as CMV pp65, is termed DNA vaccine.

Genetic immunization might be advantageous over conventional vaccination. The target DNA can be detected for a considerable period of time thus acting as a depot of the antigen. Sequence motifs in some plasmids, like GpC islands, are immunostimulatory and can function as adjuvants furthered by the immunostimulation due to LPS and other bacterial components.

Live bacterial vectors produce their own immunomodulatory factors such as lipopolysaccharides (LPS) in situ which may constitute an advantage over other forms of administration such as microencapsulation. Moreover, the use of the natural route of entry proves to be of benefit since many bacteria, like Salmonella, egress from the gut lumen via the M cells of Peyer's patches and migrate eventually into the lymph nodes and spleen, thus allowing targeting of vaccines to inductive sites of the immune system. The vaccine strain of Salmonella typhi, Ty21a, has been demonstrated to-date to have an excellent safety profile. Upon exit from the gut lumen via the M cells, the bacteria are taken up by phagocytic cells, such as macrophages and dendritic cells. These cells are activated by the pathogen and start to differentiate, and probably migrate into the lymph nodes and spleen. Due to their attenuating mutations, bacteria of the S. typhi Ty21 strain are not able to persist in these phagocytic cells but die at this time point. The recombinant DNA molecules are released and subsequently transferred into the cytosol of the phagocytic immune cells, either via a specific transport system or by endosomal leakage. Finally, the recombinant DNA molecules enter the nucleus, where they are transcribed, leading to CMV pp65 expression in the cytosol of the phagocytic cells. Specific cytotoxic T cells against CMV pp65 are induced by the activated antigen presenting cells.

There is no data available to-date indicating that S. typhi Ty21a is able to enter the bloodstream systemically. The live attenuated Salmonella typhi Ty21a vaccine strain thus allows specific targeting of the immune system while exhibiting an excellent safety profile. In contrast, adenovirus-based DNA vaccines might bear an inherent risk of unintended virus replication.

Attenuated derivatives of Salmonella enterica are attractive as vehicles for the delivery of heterologous antigens to the mammalian immune system because S. enterica strains can potentially be delivered via mucosal routes of immunization, i.e. orally or nasally, which offers advantages of simplicity and safety compared to parenteral administration. Furthermore, Salmonella strains elicit strong humoral and cellular immune responses at the level of both systemic and mucosal compartments.

In particular embodiments, the attenuated strain of Salmonella is for use in cancer immunotherapy.

In particular embodiments, cancer immunotherapy further comprises administration of one or more further attenuated strain(s) of Salmonella comprising at least one copy of a DNA molecule comprising an expression cassette encoding a tumor antigen and/or a tumor stroma antigen. In particular embodiments, said one or more further mutant strain(s) of Salmonella is/are Salmonella typhi Ty21a comprising a eukaryotic expression cassette. In particular embodiments, said one or more further strain(s) of Salmonella comprise(s) an attenuated strain of Salmonella encoding human VEGFR-2 and/or human Wilms' Tumor Protein (WT1) and/or human Mesothelin (MSLN) and/or human CEA.

Combining the attenuated strain of Salmonella of the present invention with a second attenuated strain comprising a DNA molecule encoding a second tumor antigen or a tumor stroma antigen may have synergistic antitumor effects. In particular, simultaneous targeting of different tumor antigens may minimize the risk of tumor escape. Combining CMV pp65 based cancer immunotherapy with VEGFR-2 based immunotherapy may prove especially effective, since CMV pp65 protein harboring tumor cells and the tumor vasculature are attacked at the same time.

In particular embodiments, the attenuated strain of Salmonella is co-administered with said one or more further attenuated strain(s) of Salmonella.

In the context of the present invention, the term “co-administration” or “co-administer” means administration of two different attenuated strains of Salmonella within three consecutive days, more particularly within two consecutive days, more particularly on the same day, more particularly within 12 hours. Most particularly, in the context of the present invention, the term “co-administration” refers to simultaneous administration of two different attenuated strains of Salmonella.

In particular embodiments, cancer immunotherapy is accompanied by chemotherapy, radiotherapy or biological cancer therapy. For cure of cancer, complete eradication of cancer stem cells may be essential. For maximal efficacy, a combination of different therapy approaches may be beneficial.

In the context of the present invention, the term “biological cancer therapy” refers to cancer therapy involving the use of living organisms, substances derived from living organisms, or laboratory-produced versions of such substances. Some biological therapies for cancer aim at stimulating the body's immune system to act against cancer cells (so called biological cancer immunotherapy). Biological cancer therapy approaches include the delivery of tumor antigens, delivery of therapeutic antibodies as drugs, administration of immunostimulatory cytokines and administration of immune cells. Therapeutic antibodies include antibodies targeting tumor antigens or tumor stroma antigens as well as antibodies functioning as checkpoint inhibitors, such as anti-PD-1, anti-PD-L1 and anti-CTLA4.

Chemotherapeutic agents that may be used in combination with the attenuated strain of Salmonella of the present invention may be; for example: gemcitabine, amifostine (ethyol), cabazitaxel, cisplatin, dacarbazine (DTIC), dactinomycin, docetaxel, mechlorethamine, streptozocin, cyclophosphamide, carrnustine (BCNU), lomustine (CCNU), nimustine (ACNU), doxorubicin (adriamycin), doxorubicin lipo (doxil), folinic acid, gemcitabine (gemzar), daunorubicin, daunorubicin lipo (daunoxome), procarbazine, ketokonazole, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, oxaliplatin, plicamycin, mitotane, pegaspargase, pentostatin, pipobroman, plicamycin, streptozocin, tamoxifen, temozolomide, teniposide, testolactone, thioguanine, thiotepa, uracil mustard, vinorelbine, chlorambucil and combinations thereof.

Most preferred chemotherapeutic agents according to the invention in combination with VXM65 are cabazitaxel, carboplatin, oxaliplatin, cisplatin, cyclophosphamide, docetaxel, gemcitabine, doxorubicin, paclitaxel (taxol), irinotecan, vincristine, vinblastine, vinorelbin, folinic acid, 5-fluorouracil and bleomycin, especially gemcitabine.

It may be also favorable dependent on the occurrence of possible side effects, to include treatment with antibiotics or anti-inflammatory agents.

Should adverse events occur that resemble hypersensitivity reactions mediated by histamine, leukotrienes, or cytokines, treatment options for fever, anaphylaxis, blood pressure instability, bronchospasm, and dyspnoea are available. Treatment options in case of unwanted T-cell derived auto-aggression are derived from standard treatment schemes in acute and chronic graft vs. host disease applied after stem cell transplantation. Cyclosporin and glucocorticoids are proposed as treatment options.

In the unlikely case of systemic Salmonella typhi Ty21a type infection, appropriate antibiotic therapy is recommended, for example with fluoroquinolones including ciprofloxacin or ofloxacin. Bacterial infections of the gastrointestinal tract are to be treated with respective agents, such as rifaximin.

In particular embodiments, the attenuated strain of Salmonella is administered during the chemotherapy or the radiotherapy treatment cycle or during biological cancer therapy.

In particular embodiments, the attenuated strain of Salmonella is administered before the chemotherapy or the radiotherapy treatment cycle or before biological cancer therapy. This approach may have the advantage that chemotherapy or radiotherapy can be performed under conditions of enhanced cancer immunity.

In particular embodiments, the attenuated strain of Salmonella is administered after the chemotherapy or the radiotherapy treatment cycle or after biological cancer therapy.

In particular embodiments, the attenuated strain of Salmonella is administered orally. Oral administration is simpler, safer and more comfortable than parenteral administration. In contrast, intravenous administration of live bacterial vaccines initially causes a bacteremia associated with safety risks of the sepsis-type and thus calls for careful observation and monitoring of clinical symptoms such as cytokine release. Oral administration of the attenuated strain of the present invention may at least in part overcome the described risks. However, it has to be noted that the attenuated strain of Salmonella of the present invention may also be administered by any other suitable route. Preferably, a therapeutically effective dose is administered to the subject, and this dose depends on the particular application, the type of malignancy, the subject's weight, age, sex and state of health, the manner of administration and the formulation, etc. Administration may be single or multiple, as required.

The attenuated strain of Salmonella of the present invention may be provided in the form of a solution, a suspension, lyophilisate, or any other suitable form. It may be provided in combination with pharmaceutically acceptable carriers, diluents, and/or excipients. Agents for adjusting the pH value, buffers, agents for adjusting toxicity, and the like may also be included. In the context of the present invention, the term “pharmaceutically acceptable” refers to molecular entities and other ingredients of pharmaceutical compositions that are physiologically tolerable and do not typically produce untoward reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and, more particularly, in humans.

In particular embodiments, the cancer is selected from gliomas, in particular from glioblastomas.

The vaccine of the present invention is surprisingly effective at relatively low doses. In particular embodiments, the single dose is from about 10⁵ to about 10¹¹, particularly from about 10⁶ to about 10¹⁰, more particularly from about 10⁶ to about 10⁹, more particularly from about 10⁶ to about 10⁸, most particularly from about 10⁶ to about 10⁷ colony forming units (CFU). Administration of low doses of this live bacterial vaccine minimizes the risk of excretion and thus of transmission to third parties.

In this context, the term “about” or “approximately” means within a factor of 3, alternatively within a factor of 2, including within a factor of 1.5 of a given value or range.

In particular embodiments, the attenuated strain of Salmonella is for use in individualized cancer immunotherapy comprising the step of assessing the CMV pp65 expression pattern and/or the pre-immune response against CMV pp65 of a patient.

VXM65 can be used—either by itself or in combination with other Salmonella typhi Ty21a based cancer vaccines comprising eukaryotic expression systems—for the treatment of various cancer types. In particular embodiments, VXM65 may be used for individualized patient specific cancer treatment. For that purpose, the patient's tumor and/or stromal antigen expression pattern and/or the patient's pre-immune responses against tumor and/or stromal antigens may be assessed in a first step for example by companion diagnostics targeting the patient's specific tumor and/or stromal antigen pattern. Depending on the patient's tumor and/or stromal antigen expression pattern or the patient's pre-immune responses against tumor and/or stromal antigens, VMX65 may be administered either alone or in combination with one or more suitable further Salmonella typhi Ty21a based cancer vaccine(s) comprising eukaryotic expression systems. Combinations of VXM65 with one or more further Salmonella typhi Ty21a based cancer vaccine(s) may however also be administered as fixed combinations. These cocktails combining two or more Salmonella typhi Ty21a based cancer vaccines can be composed from separate off the shelf products. The combinations, either fixed or individualized may contain VXM01 (WO 2013/091898) as anti-angiogenic basis therapy.

### SHORT DESCRIPTION OF FIGURES AND TABLES

FIG. 1: Amino acid sequence of CMV pp65 encoded by CMV pp65 cDNA contained in plasmid pVAX10.CMV65_1 (corresponding to SEQ ID NO 1)

FIG. 2: Amino acid sequence of CMV pp65 encoded by CMV pp65 cDNA contained in plasmid pVAX10.CMV65_2 (corresponding to SEQ ID NO 2)

FIG. 3: Amino acid sequence of CMV pp65 encoded by CMV pp65 cDNA contained in plasmid pVAX10.CMV65_3 (corresponding to SEQ ID NO 3)

FIG. 4: Nucleic acid sequence contained in plasmid pVAX10.CMV65_1 and encoding CMV pp65 of SEQ ID NO 1

FIG. 5: Nucleic acid sequence contained in plasmid pVAX10.CMV65_2 and encoding CMV pp65 of SEQ ID NO 2

FIG. 6: Nucleic acid sequence contained in plasmid pVAX10.CMV65_3 and encoding CMV pp65 of SEQ ID NO 3

FIG. 7: Nucleic acid sequence comprised in empty expression vector pVAX10 (pVAX10 sequence without the portion of the multiple cloning site located between restriction sites NheI and XhoI) (SEQ ID NO 7).

FIG. 8: Plasmid map of pVAX10.CMV65_1

FIG. 9: Plasmid map of pVAX10.CMV65_2

FIG. 10: Plasmid map of pVAX10.CMV65_3

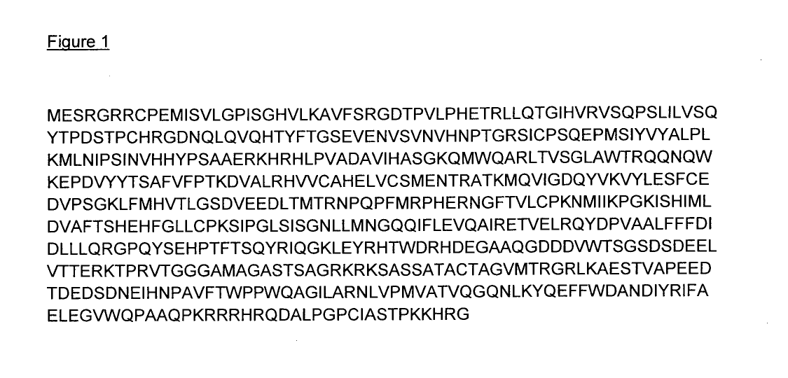

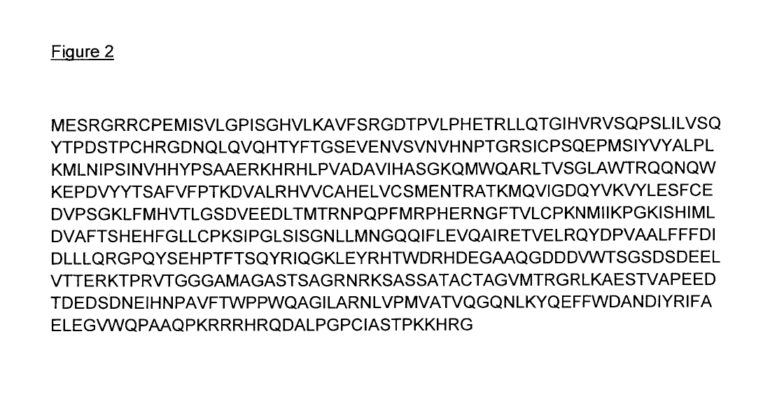

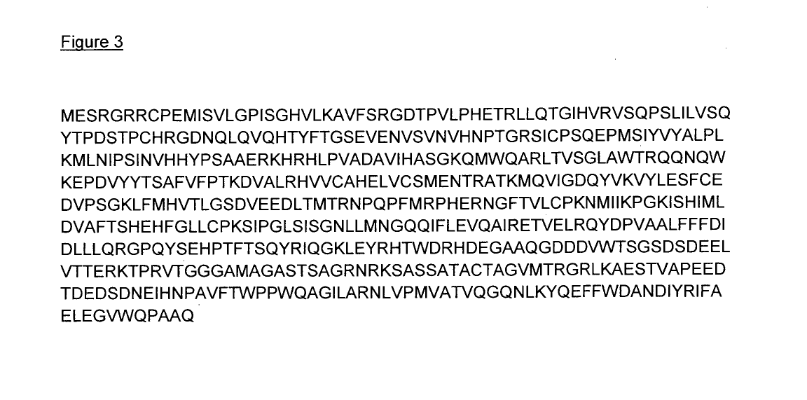

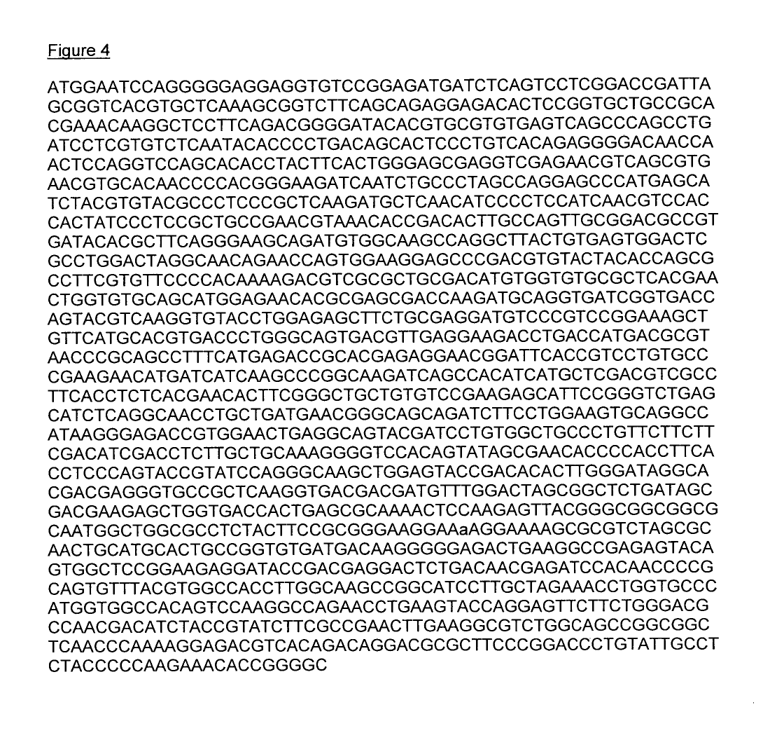

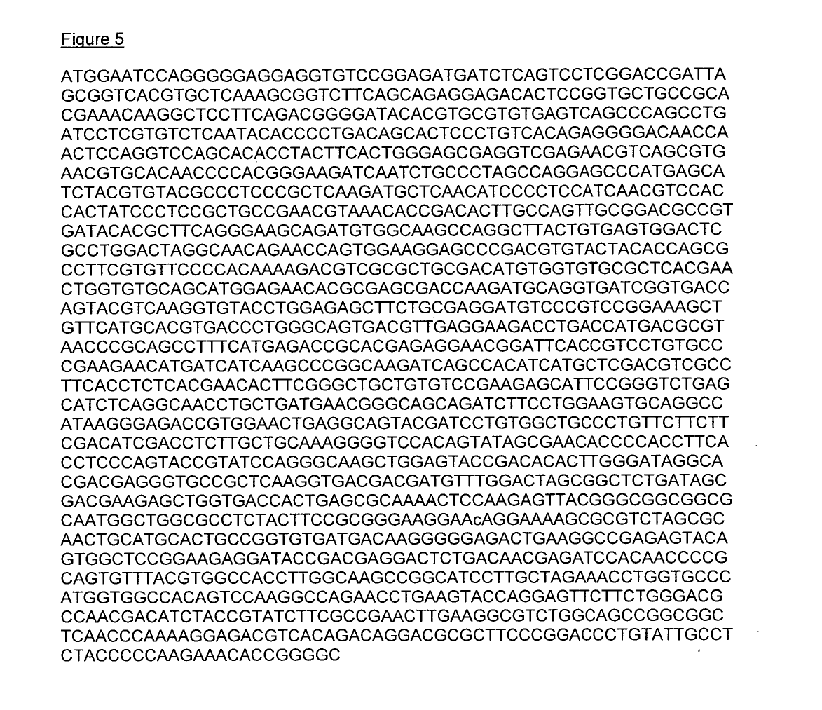

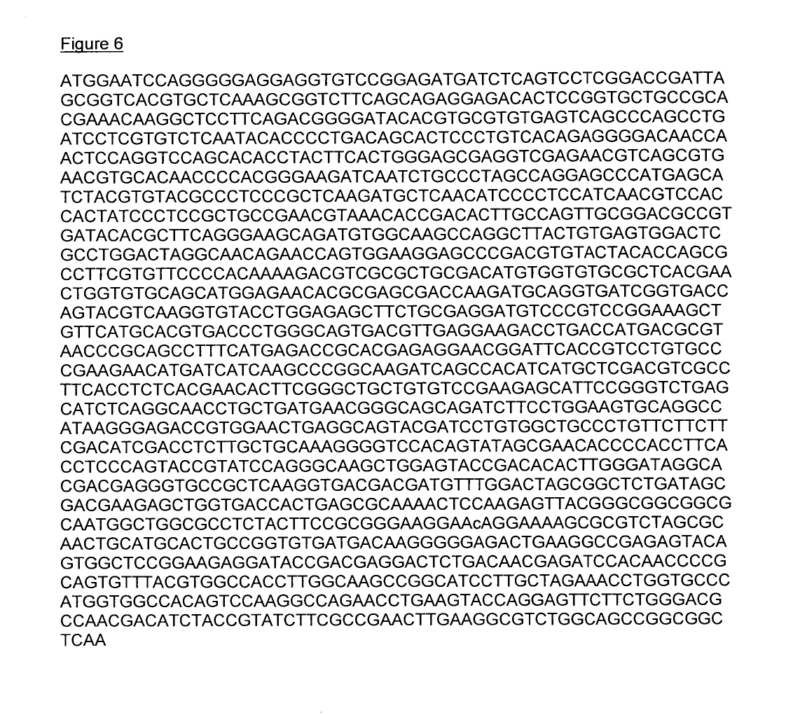

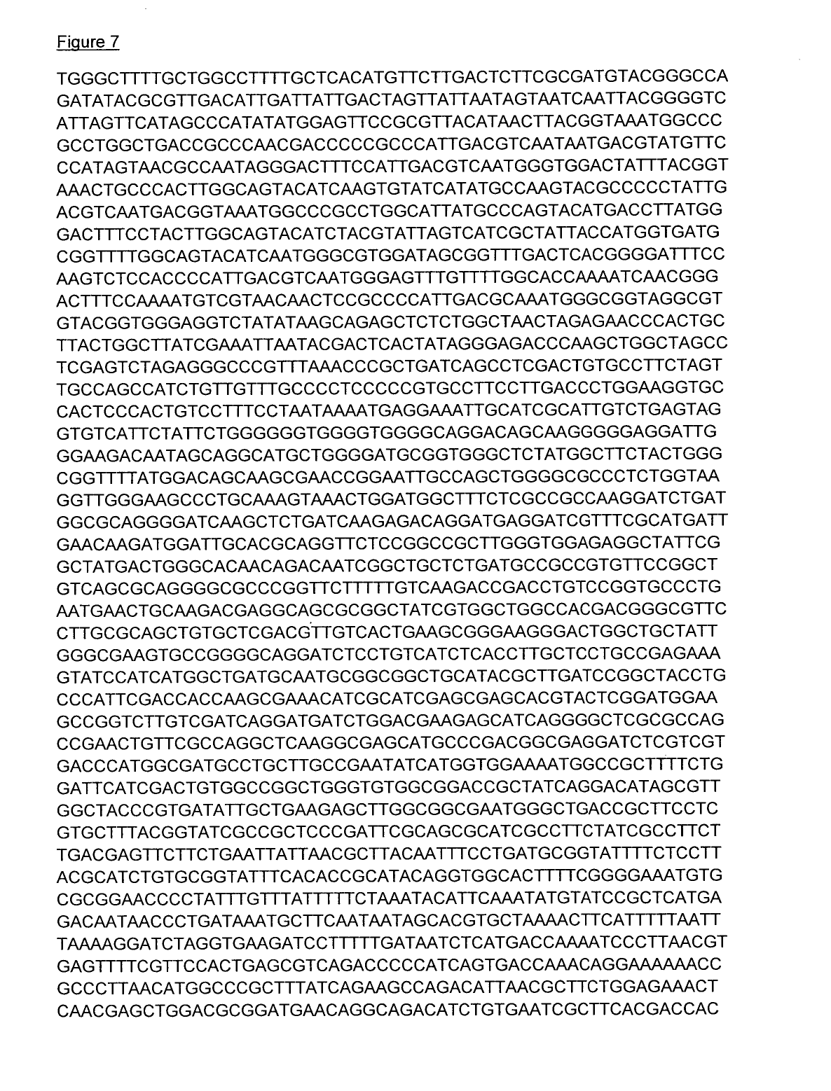

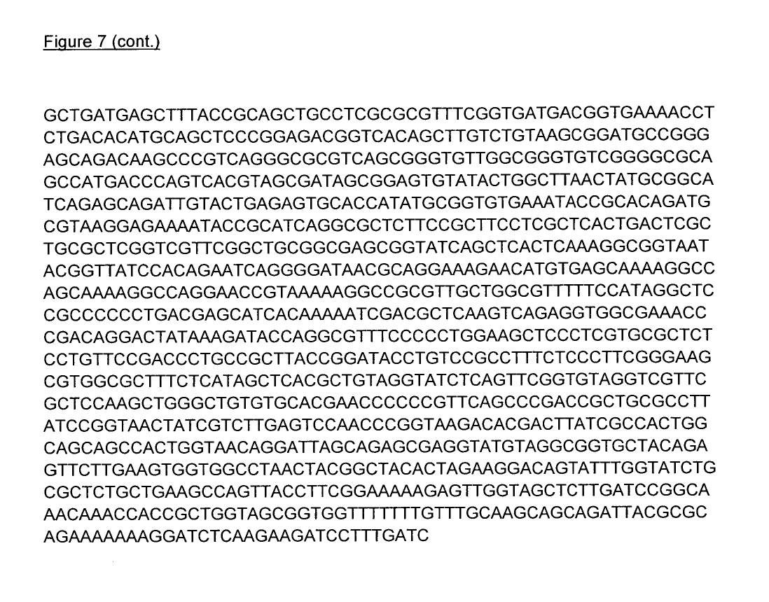

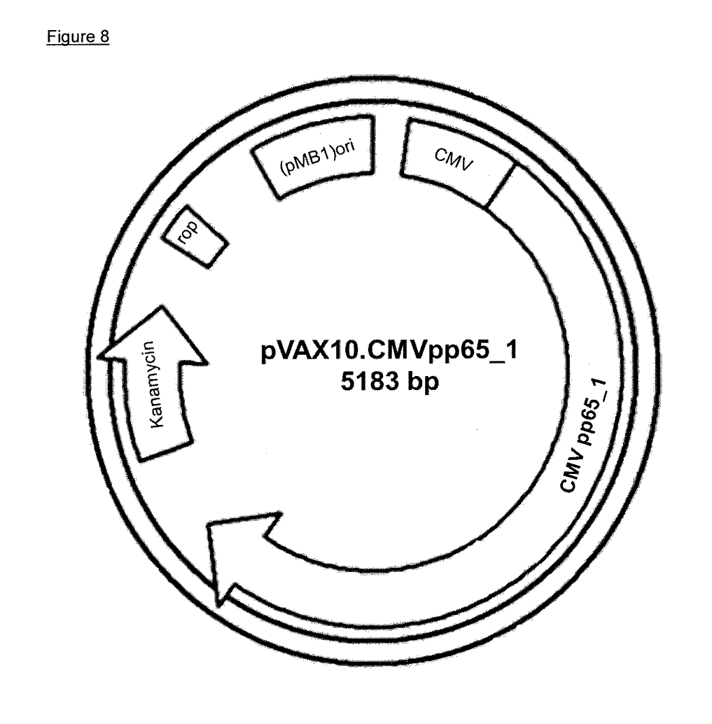

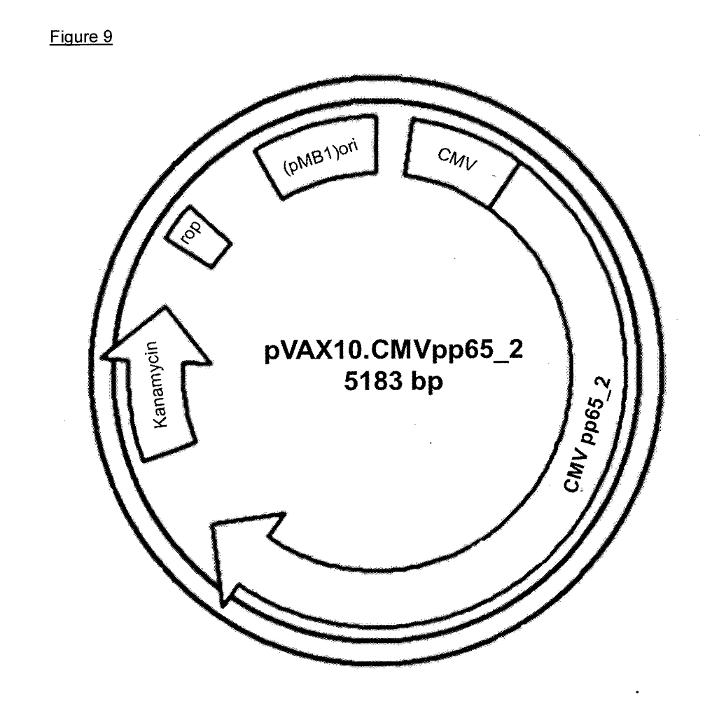

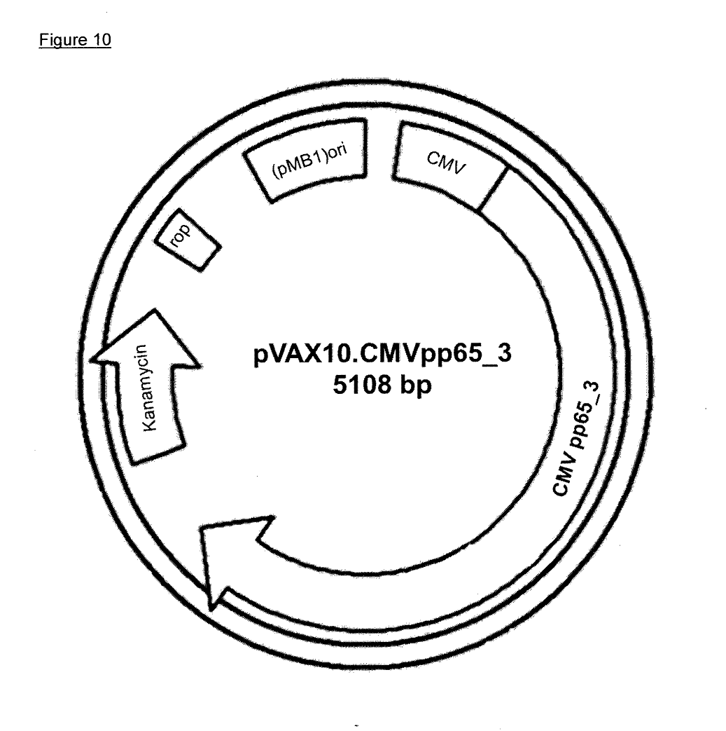

### EXAMPLES

### Example 1: Preparation of Recombinant Plasmids pVAX10.CMV65_1, pVAX10.CMV65_1 and pVAX10.CMV65_1

Three different CMV pp65 encoding cDNAs of the nucleic acid sequences SEQ ID NO 4, SEQ ID NO 5 and SEQ ID NO 6 were cloned into the pVAX10 backbone derived of pVAX10.VR2-1 (WO 2014/005683). CMV pp65 DNA fragments were generated by double-strand gene synthesis, where oligonucleotides were linked together using a thermostable ligase. The obtained ligation products were amplified by PCR. Upon amplification, the in vitro synthesized DNA fragments of CMV pp65 were cloned into the pVAX10 backbone via NheI/XhoI (the VEGFR-2 coding region of recombinant plasmid pVAX10.VR2-1 was replaced by CMV pp65). For quality control, the entire plasmids were sequenced and aligned to the respective reference sequence after transformation into E. coli. All three sequences proved to be free of errors. The resulting plasmids were designated pVAX10.CMV65_1 (containing the DNA fragment of SEQ ID NO 4), pVAX10.CMV65_2 (containing the DNA fragment of SEQ ID NO 5) and pVAX10.CMV65_3 (containing the DNA fragment of SEQ ID NO 6).

### Example 2: Transformation of Attenuated Salmonella Strains with the Recombinant Plasmids

S. typhi Ty 21a and S. typhimurium SL7207 (aroK) were transformed with plasmids pVAX10.CMV65_1, pVAX10.CMV65_2 and pVAX10.CMV65_3. The transformation was performed by electroporation.

### Preparation of Competent Salmonella Cells:

Glycerol cultures of S. typhi Ty21a and S. typhimurium SL7207 were inoculated on LB plates (animal component free [ACF] soy peptone). The plates were incubated at 37° C. overnight. One colony each was used for overnight-liquid-preculture. 3 ml LB medium (ACF soy peptone) inoculated with one colony each was incubated at 37° C. and 180 rpm overnight. To prepare competent cells, 2×300 ml of LB medium (ACF soy peptone) were inoculated with 3 ml of the overnight culture and incubated at 37° C. and 180 rpm up to an OD₆₀₀ of about 0.5. The cultures were then put on ice for 10 minutes. Subsequently, the bacteria were centrifuged for 10 minutes at 3000×g at 4° C. and each pellet was resuspended in 500 mL of ice cold H₂O_{dest}. After a new centrifugation step, the bacterial pellets were washed twice in 10% ice cold glycerol. Both pallets were put together in 2 ml of 10% glycerol and finally frozen in aliquots of 50 μL on dry ice. The used glycerol did not contain any animal ingredients (Sigma Aldrich, G5150).

### Transformation of Competent Salmonella Cells:

For each transformation reaction, a 50 μl aliquot of competent cells was thawed on ice for 10 minutes. After adding 3-5 μL of plasmid DNA the mixtures were incubated on ice for five minutes. Subsequently, the mixtures were transferred to pre-cooled cuvettes (1 mm thickness). The electric pulse was carried out at 12.5 kV/cm. Immediately afterwards, 1 ml of LB medium (ACF soy peptone) was added to the cells, the cells were transferred into a 2 ml Eppendorf tube and shaken for 1 hour at 37° C. After a short centrifugation step on a bench centrifuge (16600 rcf, 20 s), the bacterial pellet was resuspended in 200 μl of LB (ACF soy peptone) antibiotic-free medium. The mixtures were applied with a Drigalski spatula on LB plates (ACF soy peptone) containing kanamycin (concentration=25 μg/ml or 50 μg/ml). The plates were incubated at 37° C. overnight.

### Plasmid Preparation of Recombinant Salmonella Clones:

Three clones of each recombinant Salmonella strain were incubated overnight in 3 ml of LB medium (ACF soy peptone) containing kanamycin (50 μg/ml) at 37° C. The bacterial culture was then pelleted by centrifugation (16600 rcf, 30 s). Plasmid isolation was performed using the NucleoSpin Plasmid Kit from Macherey-Nagel. The plasmid DNA was eluted from the silica gel columns with 50 μl water. 5 μl of the eluate was used in agarose gel electrophoresis for control.

For long-term storage, 1 ml glycerol cultures of the positive clones were produced. For this purpose, 172 μl glycerol (no animal ingredients) was added to 828 μl medium of a logarithmically growing 3 ml culture in a 1 low ml screw microtube. The samples were stored at −70° C. until further use.

Complete Sequencing of Recombinant Plasmid DNA Isolated from Salmonella:

3 ml of liquid LB-Kan medium (ACF soy peptone) were inoculated with one colony of recombinant Salmonella and incubated overnight at 37° C. and 180 rpm. The overnight culture was pelleted by centrifugation at 1300 rpm for 30 s on a bench centrifuge (Biofuge pico, Heraeus). The plasmid isolation was performed with the NucleoSpin Plasmid Kit from Macherey-Nagel. After alkaline lysis and precipitation of high molecular weight genomic DNA and cellular components, the plasmid DNA was loaded onto columns with a silica membrane. After a washing step, the plasmids were eluted from the column with 50 μl of sterile water and sequenced. The sequences were then compared with the respective reference sequence by clone specific alignments, i.e. the plasmid sequences of each Salmonella clone was one by one aligned with the reference sequence. All sequences were in line with the respective reference sequences. The recombinant Salmonella strains were designated VXM65_1, VXM65_2, VXM65_3 (S. typhi Ty21a harboring plasmids pVAX10.CMV65_1, pVAX10.CMV65_2 and pVAX10.CMV65_3, respectively) and VXM65m_1, VXM65m_2 and VXM65m_3 (S. typhimurium SL7207 harboring plasmids pVAX10.CMV65_1, pVAX10.CMV65_2 and pVAX10.CMV65_3, respectively).

### Example 3: Preclinical Study Design—Assessing Immune Responses Elicited by VXM65 in Healthy C57Bl/6 Mice

Preclinical testing of the attenuated strain of Salmonella includes (challenge) experiments in mice where test animals are challenged with tumor cells from a stably transfected GL261 glioblastoma cell line expressing pp65. For this purpose, GL261 cells are cultured and transfected. The transfection efficiency is tested and appropriate cells are selected. The selection conditions are optimized and the cells are expanded. The desired pp65 expression is characterized and its stable expression confirmed. Four groups of C57/Bl6/6J mice (n=6 each) are challenged with a subcutaneous administration of 5×10⁵ stably transfected pp65 GL 261 glioblastoma cells on Day 0 of the study.

Three groups of animals (n=6 each) are treated with VXM65m_1, VXM65m_2 and VXM65m_3 (Salmonella typhimurium carrying CMVpp65-encoding eukaryotic expression cassettes, manufactured by VAXIMM R&D laboratory, Bad Abbach) alone at a dose of 10⁸ CFU via oral gavage on Day −7, Day −5, Day −3, and Day −1 (n=6), or with VXMO_empty vector at the same dose (n=6).

Tumor growth is measured using a micro-caliper. Animals were sacrificed as soon as tumor volume reached 1500 mm³ for animal welfare reasons.

Immune responses against CMV pp65 in healthy C57131/6 mice are evaluated by Pentamer analysis or ELISpot. Mice are vaccinated with Salmonella typhimurium containing plasmids pVAX10.CMV65_1, pVAX10.CMV65_2 and pVAX10.CMV65_3 (10¹⁰ CFU/dose). As negative control, a vector control group (10¹⁰ CFU/dose Salmonella typhimurium containing no expression plasmid) is included in the study setup to discriminate the desired immunologic effect from any unspecific background stimulation caused by Salmonella empty vector. Immune monitoring is carried out at one or more post-vaccination time points.

### 1. Animal Maintenance

Healthy female C57131/6 mice, 6 weeks old at reception, are observed for 7 days in a specific-pathogen-free (SPF) animal care unit before starting the procedure. Animals are maintained in rooms under controlled conditions of temperature (23±2° C.), humidity (45±10%), photoperiod (12 h light/12 h dark) and air exchange. Animals are maintained in SPF conditions. Room temperature and humidity are continuously monitored. The air handling system is programmed for 14 air changes/hour, with no recirculation. Fresh outside air is passed through a series of filters, before being diffused evenly into each room. A positive pressure (20±4 Pa) is maintained in the experimentation room to prevent contamination or the spread of pathogens within a rodent colony. Animals are housed in polycarbonate cages (Techniplast, Limonest, France) that are equipped to provide food and water. The standard area cages used are 800 cm² with a maximum of 10 mice per cage (from the same group). Bedding for animals is sterile corn cob bedding (ref: LAB COB 12, SERLAB, Cergy-Pontoise, France), replaced twice a week. Animal food is purchased from DIETEX (Saint-Gratien, France). Irradiated RM1 is used as sterile controlled granules. Food is provided ad libitum from water bottles equipped with rubber stoppers and sipper tubes. Water bottles are sterilized by sterile filtration and replaced twice a week. At DO, mice are distributed according to their individual body weight into 2 groups using Vivo Manager® software (Biosystemes, Couternon, France). The mean body weight of the two groups (which are then divided into groups 1 to 5 and of groups 6 to 10, respectively) is not statistically different (analysis of variance).

### 2. Treatment Schedule

The mice from groups 1 to 5 receive administrations of the vector control, the animals from groups 6 to 10 receive administrations of Salmonella typhimurium containing plasmids pVAX10.CMV65_1, pVAX10.CMV65_2 and pVAX10.CMV65_3. Both Salmonella typhimurium strains are thawed and administered within 30 min, the working solutions are discarded after use. The treatment dose is 10¹⁰ CFU in 100 μl per administration. The Salmonella strains are administered by oral gavage (per os, PO) via a cannula with a volume of 0.1 ml. Regardless of animal groups, each animal receives pre-dose application buffer to neutralize acid in the stomach prior dosing (100 μl/animal/application). This buffer is produced by dissolution of 2.6 g sodium hydrogen carbonate, 1.7 g L-ascorbic acid, 0.2 g lactose monohydrate in 100 ml of drinking water and is applied within 30 min prior application of the Salmonella typhimurium strains. The treatment schedule is as follows:

The mice from groups 1 to 5 receive daily PO administrations of vector control at 10¹⁰ in CFU every two days for four consecutive times (Q2D×4).

The mice from groups 6 to 10 receive daily PO administrations of Salmonella typhimurium containing plasmids pVAX10.CMV65_1, pVAX10.CMV65_2 and pVAX10.CMV65_3 at 10¹⁰ in CFU every two days for four consecutive times (Q2D×4).

### 3. Animal Monitoring and Termination

The viability and behavior of the animals is recorded every day, body weights are measured twice a weak.

Irrespective of the administered Salmonella vaccine, mice are terminated after 5 (groups 1 and 6), 7 (groups 2 and 7), 10 (groups 3 and 8), 14 (groups 4 and 9) and 21 (groups 5 and 10) days post vaccination phase (5 mice per animal group and time point). Isoflurane (Baxter, France) is used to anaesthetize the animals before termination. Animals are terminated by cervical dislocation. An autopsy (macroscopic examination of heart, lungs, liver, spleen, kidneys and gastrointestinal tract) is performed on all terminated animals. At the time of mice termination, spleens are collected and placed individually into single ID labeled tubes containing chilled PBS (2-8° C.) each and stored over night at 2-8° C. Freshly isolated and purified splenocytes are used for Pentamer analysis. Freshly prepared CD8+ cells are used for ELISpot analysis.

### 4. Splenocyte Preparation

Splenocyte preparation is performed as follows: In a washing step a part of the PBS is discarded and replaced by fresh PBS. A 100 μm nylon Cell Strainer (BD Falcon) is hung into the opening of a 50 ml Falcon containing 5 ml 1×PBS. The spleens are cut with a scalpel and then pushed through the cell strainer with the stamp of a 5 ml syringe. One strainer is used per spleen, the strainer is always rinsed in-between with sterile 1×PBS. The cells are centrifuged at 1,500 rpm (approximately 450 g) for 10 min at 2-8° C. and the supernatant is discarded. 1 ml ACK-Ery-Lysis buffer (8.3 g/l NH₄, 1 g/l KHCO₃, 0.037 g/l EDTA; pH 7.2-7.4) is added per spleen to lyse the red blood cells. The solution is incubated for 30 sec at RT. 10 ml of PBS are added and the cells are again spun down at 1,500 rpm for 10 min at 2-8° C., the supernatant is discarded. The pellet is resuspended in 10 ml DMEM media. Live/dead cell staining is performed with trypan blue and the cell number is counted. The cell suspension is split for the subsequent analyses. About one third is used for Pentamer analysis, the rest is used for the ELISpot analysis.

### 5. Pentamer Analysis

Pentamer Analysis includes a viability staining and the Pentamer staining. For the viability staining, one vial of the fluorescent reactive dye (Pacific Orange—component A) and the vial of anhydrous DMSO (component B) are brought to room temperature before the caps are removed. 50 μl of DMSO (component B) is added to the vial of reactive dye (component A). Subsequently the vial is mixed and it is confirmed visually that all of the dye has dissolved. The solution of reactive dye is used without delay, within a few hours of dissolution. The suspension of cells containing at least 1×10⁶ cells is centrifuged and the supernatant is discarded. The cells are washed once with 1 ml of PBS and resuspended in 1 ml of PBS. The cells are counted and the density is adjusted with PBS to 1×10⁶ cells in a 1 ml volume. 1 μl of the reconstituted fluorescent reactive dye is added to 1 ml of the cell suspension. The suspension is then mixed thoroughly and incubated at room temperature for 30 min, protected from light. The cells are washed once with 1 ml of PBS with 1% Fetal Calf Serum (FCS) and resuspended in 1 ml of PBS with 1% FCS.

For Pentamer staining, splenocytes prelabelled with Pacific Orange for viability gating are used. Recombinant glycoprotein pentamers are centrifuged in chilled microcentrifuge at 14,000×g for 5-10 minutes to collect any protein aggregates present in the solution at the bottom of the vial in order to avoid non-specific staining. The supernatant are used for Pentamer staining. All reagents are maintained on ice, shielded from light, until required. 1×10⁶ splenocytes are allocated per staining condition. The cells are washed with 2 ml wash buffer (PBS with 1% FCS) and spun down (500×g for 5 minutes), the supernatant is discarded and the cells are resuspended in the residual volume (˜50 μl). The tubes are kept chilled on ice for all subsequent steps, except where otherwise indicated. One test (2 μl) of unlabeled Pentamer is added to the cells and the solution is mixed by pipetting and incubated at room temperature (22° C.) for 10 min, shielded from light. The cells are then washed with 2 ml wash buffer and resuspended in the residual liquid (˜50 μl). Pro5® Fluorotag R-PE is spun in a chilled microcentrifuge at 14,000×g for 3 minutes to remove protein aggregates that would otherwise contribute to non-specific binding. The reagents are maintained on ice, shielded from light, until required. The supernatant is used for Pentamer staining. 8 μl Pro5® Fluorotag, 1 μl of anti-CD8 FITC and 0.5 μl anti-CD3 APC/Cy-7 antibodies are added and the solution is mixed by pipetting. The samples are incubated on ice for 20 minutes, shielded from light. The cells are washed twice with 2 ml wash buffer and each tube is mixed. 200 μl of fix solution (1% FCS, 2.5% formaldehyde in PBS) are added and the tubes are vortexed. Thorough vortexing is important to avoid cell clumping. The tubes are stored in the dark in the refrigerator until ready for data acquisition. In any case the samples are left for 3 hours before proceeding with data acquisition due to morphology changes after fixing.

Specific binding to Pentamers is investigated. Glycoprotein specific CD8⁺ T cells are counted after selecting the appropriate gates. Ratios of glycoprotein specific CD8⁺ T cells are calculated based on binding affinity to the Pentamers. The ratios are compared between the vector control and Salmonella typhimurium containing plasmids pVAX10.CMV65_1, pVAX10.CMV65_2 and pVAX10.CMV65_3 and within groups over time. A p value <0.05 is considered significant.

### 6. Antigen Expression Analysis

Antigen expression analysis is performed by transfecting plasmids pVAX10.CMV65_1, pVAX10.CMV65_2 and pVAX10.CMV65_3 into human 293T cells. 24 hours and 48 hours after infection, the cells are harvested and lysed. The obtained whole cell lysates are analyzed by SDS poly-acrylamide gel electrophoresis (SDS-PAGE), followed by Western blotting onto a PVDF membrane.

### Example 4: VXM65 Phase I Clinical Trial; Study Design

The aim of this phase I trial is to examine the safety, tolerability, and immunological responses to VXM65. The randomized, placebo-controlled, double blind dose-escalation study includes 45 subjects. The subjects receive four doses of VXM65_3 or placebo on days 1, 3, 5, and 7. Doses from 10⁶ CFU up to 10¹⁰ CFU of VXM65_3 are evaluated in the study. An independent data safety monitoring board (DSMB) is involved in the dose-escalation decisions. In addition to safety as primary endpoint, the VXM65_3-specific immune reaction are evaluated.

The objectives were to examine the safety and tolerability, and immunological responses to the investigational anti-CMV pp65 vaccine VXM65_3, as well as to identify the maximum tolerated dose (MTD) of VXM65_3. The MTD is defined as the highest dose level at which less than two of up to six patients under VXM65_3 treatment experience a dose-limiting toxicity (DLT).

Primary endpoints for safety and tolerability are adverse events and serious adverse events according to the CTCAE criteria.

Secondary endpoints, which assess the efficacy of the experimental vaccine to elicit a specific immune response to CMV pp65, include the number of immune positive patients.

VXM65_3 is manufactured according to Good Manufacturing Practice (GMP) and is given in a buffered solution. The placebo control consisted of isotonic sodium chloride solution.

The starting dose consists of a solution containing 10⁶ colony forming units (CFU) of VXM65_3 or placebo. This VXM65_3 dose was chosen for safety reasons. For comparison, one dose of Typhoral®, the licensed vaccine against typhoid fever, contains 2×10⁹ to 6×10⁹ CFU of Salmonella typhi Ty21a, equivalent to approximately thousand times the VXM65_3 starting dose. The dose is escalated in logarithmic steps, which appears to be justified for a live bacterial vaccine.

Complying with guidelines for first-in-human trials, the patients of one dose group are treated in cohorts. The first administration of VXM65_3 in any dose group is given to one patient only accompanied by one patient receiving placebo. The second cohort of each dose group consists of two patients receiving VXM65_3 and one patient receiving placebo. This staggered administration with one front-runner, i.e. only one patient receiving VXM65_3 first, serves to mitigate the risks.

A third cohort of patients (three receiving VXM65_3 and one receiving placebo) are included in the 10⁸, 10⁹, and 10¹⁰ dose groups. The third cohort and the first two cohorts of the next higher treatment group are treated in parallel based on a clearly defined randomization strategy. This strategy allows for recruitment of available patients and avoids selection bias for patients treated in parallel in the lower and higher dose group. In the 10⁶ and 10⁷ dose groups, a third cohort of patients is included only if one patient out of the initial three patients receiving VXM65_3 of the respective dose group experiences a DLT and requires confirmation by a decision of the Data Safety Monitoring Board (DSMB).

The environmental risk inherent to an oral vaccine is the potential of excretion to the environment and subsequent vaccination of people outside the target population. All study patients are confined in the study site for the period during which vaccinations take place plus three additional days. All feces of study patients are collected and incinerated. Body fluids and feces samples are investigated for VXM65_3 shedding.

Hygienic precautions are applied to protect study personnel from accidental uptake. Study personnel are trained specifically for this aspect of the study.

Patients are only discharged from hospital, if they test negative for excretion of the vaccine after the last administration of the study drug. In case a patient tests positive for excretion after the last administration, an antibiotic decontamination of the gastrointestinal tract is conducted before the patient is discharged. Excretion is followed up until results are negative. These measures appear to be justified and sufficient to protect the environment and study personnel from exposure to VXM65_3 until the shedding profile is elucidated.

In addition, specific T-cell activation and antibody formation are measured in this patient setting. A placebo control is included, in order to gain further knowledge on specific safety issues related to the active vaccine vs. the background treatment. In addition, the pooled placebo patients serve as a sound comparator for assessing specific immune activation.

### Example 5: VXM65 Specific T-Cell Responses

Responses to VXM65_3 are assessed by monitoring the frequencies of CMV pp65 specific T-cells in peripheral blood of VXM65_3 and, placebo treated patients, detected by INFγ ELISpot, at different time points prior during and post vaccination.

Firstly, T-cells and peptide pulsed DC are added to wells coated with anti-INFγ antibodies. After a period of incubation, cells are removed with secreted INFγ left binding with the coat antibodies. Then detection antibody is added to detect the bound INFγ, and after a signal amplification, the final yield can be viewed as “color spots” representing single activated and specific T-cells.

Positivity of ELISpot samples are graded according to predefined rules defining signal increase resulting in grade 0 to 3 per sample:

No increase: grade 0
Clear increase but <3×: grade 1
≥3× but <5× increase: grade 2
≥5× increase: grade 3

### Example 6: Anti-Carrier Immunity

In order to assess immune responses to the bacterial vehicle, anti-Salmonella typhi IgG and IgM immunoglobulins are detected by ELISA using two commercial assay kits (Salmonella typhi IgG ELISA, Cat. No. ST0936G and Salmonella typhi IgM ELISA, Cat. No. ST084M; Calbiotech. Inc., 10461 Austin Dr, Spring Valley, Calif. 91978, USA). These assays are qualitative assays. The assays are used as described in the package inserts respectively App. I/I) and as modified as part of the study plan according to the foregoing validation study 580.132.2785.

Both assays employ the enzyme-linked immunosorbent assay technique. Calibrator, negative control, positive control and samples are analyzed as duplicates. Diluted patient serum (dilution 1:101) is added to wells coated with purified antigen. IgG or IgM specific antibody, if present, bind to the antigen. All unbound materials are washed away and the enzyme conjugate is added to bind to the antibody-antigen complex, if present. Excess enzyme conjugate is washed off and substrate is added. The plate is incubated to allow for hydrolysis of the substrate by the enzyme. The intensity of the color generated is proportional to the amount of IgG or IgM specific antibody in the sample. The intensity of the color is measured using a spectrophotometric microtiter plate reader at 450 nm. The cut off is calculated as follows:

### Calibrator OD×Calibrator Factor (CF).

The antibody index of each determination is determined by dividing the OD value of each sample by cut-off value.

Antibody Index Interpretation:

| | |
|---|---|
| <0.9 | No detectable antibody to Salmonella typhi IgG or IgM by |
| | ELISA |
| 0.9-1.1 | Borderline positive |
| >1.1 | Detectable antibody to Salmonella typhi IgG or IgM by ELISA |

### Example 7: Excretion

The shedding of bacteria in stool and body fluids, tears, saliva, urine and blood is monitored in the study according to methods validated transferred as formerly validated according to GLP at an established central service laboratory (Huntingdon Life Sciences, Huntingdon, UK). Shedding and biodistribution in body fluids of VXM65_3 are determined by plate and enrichment cultivation. Identity of the VXM65_3 carrier bacterium is determined by serological agglutination and PCR methods.

1. An attenuated strain of Salmonella comprising at least one copy of a DNA molecule comprising an expression cassette encoding CMV pp65.

2. The attenuated strain of Salmonella of claim 1, wherein the attenuated strain of Salmonella is of the species Salmonella enterica, particularly wherein the attenuated strain of Salmonella is Salmonella typhi Ty21a.

3. The attenuated strain of Salmonella of claim 1 or 2, wherein the expression cassette is a eukaryotic expression cassette.

4. The attenuated strain of Salmonella of any one of claims 1 to 3, wherein CMV pp65 is selected from the group consisting of CMV pp65 having the amino acid sequence as found in SEQ ID NO 1 and a protein that shares at least 80% sequence identity therewith, CMV pp65 having the amino acid sequence as found in SEQ ID NO 2 and a protein that shares at least 80% sequence identity therewith, and CMV pp65 having the amino acid sequence as found in SEQ ID NO 3 and a protein that shares at least 80% sequence identity therewith, particularly wherein CMV pp65 has the amino acid sequence as found in SEQ ID NO 1, in SEQ ID NO 2 or in SEQ ID NO 3.

5. The attenuated strain of Salmonella of claim 3 or 4, wherein the DNA molecule comprises the kanamycin antibiotic resistance gene, the pMB1 ori, and a eukaryotic expression cassette encoding CMV pp65 having the amino acid sequence as found in SEQ ID NO 1 or a protein that shares at least 80% sequence identity therewith, CMV pp65 having the amino acid sequence as found in SEQ ID NO 2 or a protein that shares at least 80% sequence identity therewith, or CMV pp65 having the amino acid sequence as found in SEQ ID NO 3 or a protein that shares at least 80% sequence identity therewith, under the control of a CMV promoter, particularly wherein CMV pp65 has the nucleic acid sequence as found in SEQ ID NO 4, in SEQ ID NO 5 or in SEQ ID NO 6.

6. The attenuated strain of Salmonella of any one of claims 1 to 5 for use as a medicament.

7. The attenuated strain of Salmonella of claim 6 for use as a vaccine.

8. The attenuated strain of Salmonella of claim 7 for use in cancer immunotherapy.

9. The attenuated strain of Salmonella of claim 8, wherein cancer immunotherapy further comprises administration of one or more further attenuated strain(s) of Salmonella comprising at least one copy of a DNA molecule comprising an expression cassette encoding a tumor antigen and/or a tumor stroma antigen, particularly wherein said one or more further attenuated strain(s) of Salmonella is/are Salmonella typhi Ty21a comprising a eukaryotic expression cassette, more particularly wherein said one or more further attenuated strain(s) of Salmonella comprise(s) an attenuated strain of Salmonella encoding human VEGFR-2 and/or human Wilms' Tumor Protein (WT1) and/or human Mesothelin (MSLN) and/or human CEA.

10. The attenuated strain of Salmonella of any of the preceding claims, in particular according to claim 9, wherein the attenuated strain of Salmonella is co-administered with said one or more further attenuated strain(s) of Salmonella.

11. The attenuated strain of Salmonella of any one of claims 8 to 10, wherein cancer immunotherapy is accompanied by chemotherapy, radiotherapy or biological cancer therapy, particularly wherein the attenuated strain of Salmonella is administered before or during the chemotherapy or the radiotherapy treatment cycle or before or during biological cancer therapy, or before and during the chemotherapy or the radiotherapy treatment cycle or the biological cancer therapy.

12. The attenuated strain of Salmonella of any one of claims 6 to 11, wherein the attenuated strain of Salmonella is administered orally.

13. The attenuated strain of Salmonella of any one of claims 8 to 12, wherein the cancer is selected from gliomas, in particular from glioblastomas.

14. The attenuated strain of Salmonella of any one of claims 6 to 13, wherein the single dose comprises from about 10⁵ to about 10¹¹, particularly from about 10⁶ to about 10¹⁰, more particularly from about 10⁶ to about 10⁹, more particularly from about 10⁶ to about 10⁸, most particularly from about 10⁶ to about 10⁷ colony forming units (CFU).

15. The attenuated strain of Salmonella of any one of claims 8 to 14 for use in individualized cancer immunotherapy comprising the step of assessing the CMV pp65 expression pattern and/or the pre-immune response against CMV pp65 of a patient.


SEQUENCE LISTING (7 sequences)

SEQ ID NO: 1 - protein, 561 aa, CMV
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 2 - protein, 561 aa, artificial
  mutated CMV pp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 3 - protein, 536 aa, artificial
  mutated CMV pp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQ

SEQ ID NO: 4 - DNA, 1683 bp, CMV
atggaatccagggggaggaggtgtccggagatgatctcagtcctcggaccgattagcggtcacgtgctcaaagcggtcttcagcagaggagacactccggtgctgccgcacgaaacaaggctccttcagacggggatacacgtgcgtgtgagtcagcccagcctgatcctcgtgtctcaatacacccctgacagcactccctgtcacagaggggacaaccaactccaggtccagcacacctacttcactgggagcgaggtcgagaacgtcagcgtgaacgtgcacaaccccacgggaagatcaatctgccctagccaggagcccatgagcatctacgtgtacgccctcccgctcaagatgctcaacatcccctccatcaacgtccaccactatccctccgctgccgaacgtaaacaccgacacttgccagttgcggacgccgtgatacacgcttcagggaagcagatgtggcaagccaggcttactgtgagtggactcgcctggactaggcaacagaaccagtggaaggagcccgacgtgtactacaccagcgccttcgtgttccccacaaaagacgtcgcgctgcgacatgtggtgtgcgctcacgaactggtgtgcagcatggagaacacgcgagcgaccaagatgcaggtgatcggtgaccagtacgtcaaggtgtacctggagagcttctgcgaggatgtcccgtccggaaagctgttcatgcacgtgaccctgggcagtgacgttgaggaagacctgaccatgacgcgtaacccgcagcctttcatgagaccgcacgagaggaacggattcaccgtcctgtgcccgaagaacatgatcatcaagcccggcaagatcagccacatcatgctcgacgtcgccttcacctctcacgaacacttcgggctgctgtgtccgaagagcattccgggtctgagcatctcaggcaacctgctgatgaacgggcagcagatcttcctggaagtgcaggccataagggagaccgtggaactgaggcagtacgatcctgtggctgccctgttcttcttcgacatcgacctcttgctgcaaaggggtccacagtatagcgaacaccccaccttcacctcccagtaccgtatccagggcaagctggagtaccgacacacttgggataggcacgacgagggtgccgctcaaggtgacgacgatgtttggactagcggctctgatagcgacgaagagctggtgaccactgagcgcaaaactccaagagttacgggcggcggcgcaatggctggcgcctctacttccgcgggaaggaaaaggaaaagcgcgtctagcgcaactgcatgcactgccggtgtgatgacaagggggagactgaaggccgagagtacagtggctccggaagaggataccgacgaggactctgacaacgagatccacaaccccgcagtgtttacgtggccaccttggcaagccggcatccttgctagaaacctggtgcccatggtggccacagtccaaggccagaacctgaagtaccaggagttcttctgggacgccaacgacatctaccgtatcttcgccgaacttgaaggcgtctggcagccggcggctcaacccaaaaggagacgtcacagacaggacgcgcttcccggaccctgtattgcctctacccccaagaaacaccggggc

SEQ ID NO: 5 - DNA, 1683 bp, artificial
  mutated CMV pp65 cDNA
atggaatccagggggaggaggtgtccggagatgatctcagtcctcggaccgattagcggtcacgtgctcaaagcggtcttcagcagaggagacactccggtgctgccgcacgaaacaaggctccttcagacggggatacacgtgcgtgtgagtcagcccagcctgatcctcgtgtctcaatacacccctgacagcactccctgtcacagaggggacaaccaactccaggtccagcacacctacttcactgggagcgaggtcgagaacgtcagcgtgaacgtgcacaaccccacgggaagatcaatctgccctagccaggagcccatgagcatctacgtgtacgccctcccgctcaagatgctcaacatcccctccatcaacgtccaccactatccctccgctgccgaacgtaaacaccgacacttgccagttgcggacgccgtgatacacgcttcagggaagcagatgtggcaagccaggcttactgtgagtggactcgcctggactaggcaacagaaccagtggaaggagcccgacgtgtactacaccagcgccttcgtgttccccacaaaagacgtcgcgctgcgacatgtggtgtgcgctcacgaactggtgtgcagcatggagaacacgcgagcgaccaagatgcaggtgatcggtgaccagtacgtcaaggtgtacctggagagcttctgcgaggatgtcccgtccggaaagctgttcatgcacgtgaccctgggcagtgacgttgaggaagacctgaccatgacgcgtaacccgcagcctttcatgagaccgcacgagaggaacggattcaccgtcctgtgcccgaagaacatgatcatcaagcccggcaagatcagccacatcatgctcgacgtcgccttcacctctcacgaacacttcgggctgctgtgtccgaagagcattccgggtctgagcatctcaggcaacctgctgatgaacgggcagcagatcttcctggaagtgcaggccataagggagaccgtggaactgaggcagtacgatcctgtggctgccctgttcttcttcgacatcgacctcttgctgcaaaggggtccacagtatagcgaacaccccaccttcacctcccagtaccgtatccagggcaagctggagtaccgacacacttgggataggcacgacgagggtgccgctcaaggtgacgacgatgtttggactagcggctctgatagcgacgaagagctggtgaccactgagcgcaaaactccaagagttacgggcggcggcgcaatggctggcgcctctacttccgcgggaaggaacaggaaaagcgcgtctagcgcaactgcatgcactgccggtgtgatgacaagggggagactgaaggccgagagtacagtggctccggaagaggataccgacgaggactctgacaacgagatccacaaccccgcagtgtttacgtggccaccttggcaagccggcatccttgctagaaacctggtgcccatggtggccacagtccaaggccagaacctgaagtaccaggagttcttctgggacgccaacgacatctaccgtatcttcgccgaacttgaaggcgtctggcagccggcggctcaacccaaaaggagacgtcacagacaggacgcgcttcccggaccctgtattgcctctacccccaagaaacaccggggc

SEQ ID NO: 6 - DNA, 1608 bp, artificial
  mutated CMV pp65 cDNA
atggaatccagggggaggaggtgtccggagatgatctcagtcctcggaccgattagcggtcacgtgctcaaagcggtcttcagcagaggagacactccggtgctgccgcacgaaacaaggctccttcagacggggatacacgtgcgtgtgagtcagcccagcctgatcctcgtgtctcaatacacccctgacagcactccctgtcacagaggggacaaccaactccaggtccagcacacctacttcactgggagcgaggtcgagaacgtcagcgtgaacgtgcacaaccccacgggaagatcaatctgccctagccaggagcccatgagcatctacgtgtacgccctcccgctcaagatgctcaacatcccctccatcaacgtccaccactatccctccgctgccgaacgtaaacaccgacacttgccagttgcggacgccgtgatacacgcttcagggaagcagatgtggcaagccaggcttactgtgagtggactcgcctggactaggcaacagaaccagtggaaggagcccgacgtgtactacaccagcgccttcgtgttccccacaaaagacgtcgcgctgcgacatgtggtgtgcgctcacgaactggtgtgcagcatggagaacacgcgagcgaccaagatgcaggtgatcggtgaccagtacgtcaaggtgtacctggagagcttctgcgaggatgtcccgtccggaaagctgttcatgcacgtgaccctgggcagtgacgttgaggaagacctgaccatgacgcgtaacccgcagcctttcatgagaccgcacgagaggaacggattcaccgtcctgtgcccgaagaacatgatcatcaagcccggcaagatcagccacatcatgctcgacgtcgccttcacctctcacgaacacttcgggctgctgtgtccgaagagcattccgggtctgagcatctcaggcaacctgctgatgaacgggcagcagatcttcctggaagtgcaggccataagggagaccgtggaactgaggcagtacgatcctgtggctgccctgttcttcttcgacatcgacctcttgctgcaaaggggtccacagtatagcgaacaccccaccttcacctcccagtaccgtatccagggcaagctggagtaccgacacacttgggataggcacgacgagggtgccgctcaaggtgacgacgatgtttggactagcggctctgatagcgacgaagagctggtgaccactgagcgcaaaactccaagagttacgggcggcggcgcaatggctggcgcctctacttccgcgggaaggaacaggaaaagcgcgtctagcgcaactgcatgcactgccggtgtgatgacaagggggagactgaaggccgagagtacagtggctccggaagaggataccgacgaggactctgacaacgagatccacaaccccgcagtgtttacgtggccaccttggcaagccggcatccttgctagaaacctggtgcccatggtggccacagtccaaggccagaacctgaagtaccaggagttcttctgggacgccaacgacatctaccgtatcttcgccgaacttgaaggcgtctggcagccggcggctcaa

SEQ ID NO: 7 - DNA, 3500 bp, artificial
  expression plasmid
tgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagacccccatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatc